(19) United States
(12) Patent Application Publication
(10) Pub. No.: US 2014/0010710 A1
(43) Pub. Date: Jan. 9, 2014

(54) DESTRUCTION OF GENETIC MATERIAL

(71) Applicants: Brian G. Larson, Alpine, UT (US); David Larson, Pleasant Grove, UT (US)

(72) Inventors: Brian G. Larson, Alpine, UT (US); David Larson, Pleasant Grove, UT (US)

(21) Appl. No.: 13/705,454

(22) Filed: Dec. 5, 2012

Related U.S. Application Data
(60) Provisional application No. 61/566,936, filed on Dec. 5, 2011.

(51) Int. Cl. A61L 2/16

(52) U.S. Cl. CPC A61L 2/16
USPC 422/28

(57) ABSTRACT

The present disclosure is drawn to methods of destroying genetic material in laboratory environments. Steps include contacting a surface in a genetic laboratory that is contaminated with genetic material with a decontaminant composition comprising a transition metal or alloy thereof, an alcohol, a peroxygen, and water; and maintaining contact between the decontaminant composition and the surface for a sufficient period of time to provide a reduction in genetic material on the surface.

### BACKGROUND

A commonly used decontaminant for removing or destroying genetic material from working surfaces and instruments in forensic or other laboratories is chlorine bleach solution, e.g., 10 wt % bleach. This diluted solution is usually made daily, applied to surfaces for a period of time to destroy as much genetic material as possible, and then is typically wiped away. While bleach solution has been shown to be somewhat effective in removing or destroying genetic material, there are certain drawbacks. First, it would be desirable to improve the level of DNA reduction, particularly when the genetic material is present in a biological fluid. Second, bleach is known to be hazardous to humans, emitting undesirable fumes that can cause asthma and other respiratory harm, and furthermore, direct contact with bleach can be damaging to the skin, eyes, mucosal surfaces, etc. Third, diluted bleach loses its effectiveness after 24 hours. As a result of it's relatively short time of effectiveness after dilution, the use of bleach solution requires extra preparation effort, which can put a strain on laboratory personnel to keep up with this task of preparing appropriate bleach solutions for decontamination. As another complication, even undiluted bleach will begin to degrade after being stored for 6 months. Apart from the poor shelf life of diluted bleach, this substance is also corrosive, and thus, when used on laboratory surfaces or instruments, it should be followed up with the application of distilled or purified water to stop the formation of crystals and to reduce its corrosive effects.

### SUMMARY

It has been recognized that it would be desirable to provide methods of destroying genetic material in a laboratory environment, and in one embodiment, without the need for the use of corrosive material such as bleach. The method can comprise steps of contacting a surface that is contaminated with genetic material with a decontaminant composition. The decontaminant composition can comprise a transition metal or alloy thereof, an alcohol, a peroxygen, and water. An additional step includes maintaining contact between the decontaminant composition and the surface for a sufficient period of time to provide a reduction in genetic material on the surface. An additional optional step can include wiping the surface to remove used decontaminant composition (and residual genetic material) from the surface. This may provide the benefit of avoiding possible false data being generated during subsequent genetic testing due to the presence of leftover decontaminant composition or residual genetic material that was not destroyed during the process.

In one example, the decontaminant composition can be prepared by admixing at least two parts together in accordance with a preliminary step of admixing a first liquid composition and a second liquid composition to form the decontaminant composition. The first liquid composition can comprise the transition metal or alloy thereof and the alcohol, and the second liquid composition can comprise the peroxygen and water.

Additional features and advantages of the disclosure will be apparent from the detailed description that follows, which illustrates, by way of example, features of the disclosure.

### DETAILED DESCRIPTION

Reference will now be made to the exemplary embodiments, and specific language will be used herein to describe the same. It will nevertheless be understood that no limitation of the scope of the disclosure is thereby intended. Alterations and further modifications of the inventive features illustrated herein, and additional applications of the principles of the disclosures as illustrated herein, which would occur to one skilled in the relevant art and having possession of this disclosure, are to be considered within the scope of the disclosure. It is also to be understood that the terminology used herein is used for the purpose of describing particular embodiments only. The terms are not intended to be limiting unless specified as such.

It is noted that, as used in this specification and the appended claims, the singular forms “a,” “an,” and “the” include plural referents unless the content clearly dictates otherwise.

The term “genetic material” refers to any hereditary information, such as in DNA or RNA, including both the genes and the non-coding sequences of the DNA or RNA.

The term “laboratory” or “laboratory environment” refers to any location where genetic testing or other activity is conducted, ranging in size from a full genetic, medical, or forensic laboratory environment to small portable equipment used for collecting, assaying, storing, transporting, etc., genetic material. Thus, in addition to a traditional clean room laboratory, mobile equipment used outside of a traditional lab site is also considered to be a “laboratory” in accordance with examples of the present disclosure, provided the laboratory includes equipment with surfaces where genetic material is collected, sampled, stored, assayed, etc., for purposes of genetic testing, collection, or transport.

The term “solution” is also used throughout the specification to describe the decontaminant compositions of the present disclosure. However, as these “solutions” can include colloidal transition metals, these compositions can also be described as dispersions or suspensions. As the continuous phase is typically a solution, and the transition metal can be present in ionic and/or colloidal form (and typically in small amounts and sizes), for convenience, these compositions will typically be referred to as “solutions” herein. Further, sometimes a decontaminant solution is referred to as a “resultant” decontaminant solution. This is to provide added clarity that the decontaminant solution is a product of the mixing of the two-part systems. This being stated, the terms “decontaminant solution” and “resultant decontaminant solution” can be used interchangeably herein as is typically clear from the context of the discussion.

The term “substantially free” when used with regard to the decontaminant compositions of the present disclosure refers to the total absence of or near total absence of a specific compound or composition. For example, when a composition is said to be substantially free of aldehydes, there are either no aldehydes in the composition or only trace amounts of aldehydes in the composition.

The term “peroxygen” refers to any compound containing a dioxygen (O—O) bond. Dioxygen bonds, particularly bivalent O—O bonds, are readily cleavable, thereby allowing compounds containing them to act as powerful oxidizers. Non-limiting examples of classes of peroxygen compounds include peracids, peracid salts, and peroxides such as hydrogen peroxide or metal peroxides.

When referring to the term “alloy,” it is understood that individual colloidal or metallic particles can be in the form of composites of multiple metals, or alloys can also include co-dispersions of multiple metals as separate particles.

The term “two-part” when referring to the systems of the present disclosure is not limited to systems having only two parts. For example, the system can be a concentrate, and thus, is actually a three part system, e.g., a first part including transition metal and alcohol, a second part including a peroxygen and water, and a third part of a diluting solvent for diluting the first part, the second part, and/or the resultant decontaminant solution. Non-limiting examples of diluting solvents include water, alcohols, or combinations thereof. When the diluting solvent is an alcohol, it can, but need not be the same alcohol or mixture of alcohols which are present in the first “part” of the system. Thus, “two-part’ is specifically defined herein to mean, at least two parts, unless the context dictates otherwise.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight ratio range of about 1 wt % to about 20 wt % should be interpreted to include not only the explicitly recited limits of 1 wt % and about 20 wt %, but also to include individual weights such as 2 wt %, 11 wt %, 14 wt %, and sub-ranges such as 10 wt % to 20 wt %, 5 wt % to 15 wt %, etc.

In accordance with this, methods of destroying genetic material in a laboratory environment, such as a genetic or medical laboratory, can comprise steps of contacting a surface in a laboratory with a decontaminant composition that is contaminated with genetic material. The decontaminant composition can comprise a transition metal or alloy thereof, an alcohol, a peroxygen, and water. An additional step includes maintaining contact between the decontaminant composition and the surface for a sufficient period of time to provide a reduction in genetic material on the surface. An additional optional step can include wiping the surface to remove used decontaminant composition (and/or residual genetic material) from the surface. This may provide the benefit of avoiding possible false data being generated during subsequent genetic testing due to the presence of leftover decontaminant composition or residual genetic material that was not destroyed during the process. Wiping step(s) can be carried out after the contacting step, but before the maintaining step, or alternatively, after the maintaining step, e.g., after achieving the reduction in genetic material on the surface, e.g., at least a 1-fold reduction, at least 3-fold reduction, at least 10-fold reduction, at least 40-fold reduction, etc. Additionally, in one example, multiple wiping steps may be carried out, such as an initial wiping step to remove used decontaminant composition and residual genetic material, followed by a subsequent wiping step with water or another solvent. Any combination of wiping steps can be carried out as would be apparent to one skilled in the art after considering the present disclosure.

In one specific example, the decontaminant composition can be prepared by admixing at least two parts together in accordance with a preliminary step of admixing a first liquid composition and a second liquid composition to form the decontaminant composition. The first liquid composition can comprise the transition metal or alloy thereof and the alcohol, and the second liquid composition can comprise the peroxygen and the water. In these “two-part” embodiments, it is notable that the concentrations of each ingredient can be described in the context of concentration in the first or second liquid composition, or the resultant decontaminant solution. The concentration of a compound in the first or second liquid composition will usually be lower in the resultant decontaminant solution than in the first or second liquid composition, as the amount typically gets diluted by the other part of the system. This being stated, this is not always the case, depending on the ingredients in the other portion of the two-part system.

When a two part solution is brought together, reactions occur that can also reduce or increase relative concentrations of given ingredients, e.g., in the case of peracid/peroxide compositions, the peroxide component of the peracid is rapidly converted into water and oxygen within minutes of activation, and ceases to exist in some cases. Additionally, such two-part embodiments provide effective decontamination for a period of weeks, e.g., up to 60 days after activation or more, depending on the specific composition. Furthermore, whether two part system or a single solution, these compositions can be prepared so that they are non-corrosive or non-toxic, emit no emissions into the environment, and can provide long-term cost savings because of reduction in labor and replacement parts. Furthermore, these solutions can be prepared so that they pose no health or safety issues, since all of the ingredients are essentially food grade after activation. For example, in the case of some two part systems of peracids and peroxides, e.g., peroxyacetic acid and hydrogen peroxide, after activation by bringing the two parts together, the dramatically altered chemical form of the peracid post-activation is no longer corrosive to the skin, exhibits no oral or inhalation toxicities, no dermal toxicities, and only mild irritation when sprayed directly into the eyes (no permanent damage to the eyes). Furthermore, the composition is no longer a highly-oxidizing agent to materials and is safe for direct application on stainless steels, plastics, and polymers exhibiting excellent inhibition corrosion rates (unlike bleach).

In accordance with examples of the present disclosure, the methods described herein are particularly useful for decontaminating hard surfaces, including laboratory workbench surfaces, laboratory equipment, and the like. The compositions are particularly useful in decontaminating surfaces that may have genetic material thereon in preparation for subsequent usage. Any technique using a DNA source that needs to be destroyed can benefit from the present methods, including blood (whole blood, plasma, etc.), amplified DNA, extracted DNA, RNA, etc., including both the genes and the non-coding sequences of the DNA or RNA.

In certain examples of the present disclosure, the decontaminant composition can be applied to the surface at least at a 1:1 weight ratio of decontaminant composition to genetic material sample. This may work well particularly for amplified DNA, extracted DNA, or other forms of exposed DNA or RNA. With encased DNA, as would be typical with DNA present in bodily fluids, e.g., blood, semen, saliva, mucous, etc., greater ratios of decontaminant composition to genetic material sample may be more useful, e.g., at least 2:1, at least 3:1, at least 5:1, at least 10:1, etc., though these ranges are not intended to be limiting.

Laboratories that can be decontaminated with the methods described herein are diverse, with a linking characteristic being the use of surfaces or instrumentation where genetic testing, storage, transport, etc., may be desired. Examples of so-called laboratories can include forensics laboratories, medical laboratories, mobile laboratories, lab on a chip systems, sampling or transport containers, or the like.

Turning to the compositional components more specifically, the alcohol can be present in the first liquid composition at from about 0.005 wt % to 99.99 wt %, with the upper end of the range being modifiable to 80 wt % or 50 wt %, and the lower end of the range being modifiable to 0.05 wt % or 0.1 wt %. This being stated, it is also noted that in certain embodiments, the alcohol can be present in the resultant decontaminant solution at from 0.001 wt % to 95 wt %, with the lower end of the range being modifiable to 0.05 wt % or 0.1 wt %, and the upper end of the range being modifiable to 40 wt %, 30 wt %, 20 wt % or 10 wt % in accordance with embodiments of the present disclosure. Any combination of these upper and lower limits is included herein.

Regarding the transition metal or alloy present in the first liquid composition, the range of 0.0005 ppm to 100,000 ppm by weight can be modified at the upper end of the range to 20,000 ppm or 5,000 ppm, and/or can be modified at the lower end of the range to 0.001 ppm, 0.01 ppm, or 1 ppm. The resultant decontaminant solution, on the other hand, can include from 0.001 ppm to 50,000 ppm by weight of the transition metal or alloy thereof. This range can be modified at the upper end of the range to 10,000 ppm, 5,000 ppm, or 1,500 ppm, and at the lower end of the range from 0.1, 1, or 15, for example. Any combination of these upper and lower limits is included herein.

Regarding the second liquid composition, water and the peroxygen compound can be present at various ratios. For example, the peroxygen can be present in the second liquid composition at from 0.001 wt % to 80 wt %, with the upper end of the range being modifiable to 30 wt % or 15 wt %, and the lower end of the range being modifiable to 0.01 wt % or 0.05 wt %. Regarding the resultant decontaminant solution, the peroxygen content can be, for example, from 0.01 wt % to 20 wt %, with the upper end of the range being modifiable to 10 wt %, 5 wt %, or 2 wt %, and the lower end of the range being modifiable to 0.01 wt %, 0.2 wt %, or 0.3 wt %. Again, any combination of these upper and lower limits is included herein.

As these ranges are merely exemplary, one skilled in the art could modify these ranges for a particular application, considering such things as the type of alcohol (polyhydric, food grade, mixtures, etc.); the type of peroxygen (peroxide, peracid, combination of peroxide/peracid, etc.); and the type of metal (ionic, colloidal, alloy, etc.).

The compositions used for the methods described herein can be formulated and packaged in any manner known to those skilled in the art. In one embodiment, a pre-mixed composition can be prepared and shipped to the user, or alternatively, two liquid compositions can be contained in separate containers such as bottles, jars, bags, dispensers, etc., to be combined prior to use by the end user. In one aspect of the disclosure, liquid compositions can be formulated so that the decontaminant solution can be made from the two liquid compositions alone, or the two liquid compositions of the system can be formulated to provide a concentrate of the decontaminant solution which can be diluted to a desired decontaminant potency level with water or other diluting solvent(s). Furthermore, in two-part systems, the two liquid compositions of the system can be placed in separate compartments of a single container.

In one embodiment, the decontaminant solution (one part or multiple parts) can include only ingredients that are food-grade or food safe. For example, though not required, the composition can be substantially free of decontaminant ingredients commonly present in many commercially available surface cleaners. Examples of non-food-grade ingredients which can be omitted from the decontaminant solution include, but are not limited to, aldehydes such as glutaraldehyde; chlorine-based decontaminants; chlorine and bromine-based decontaminants; iodophore-based decontaminants; phenolic-based decontaminants, quaternary ammonium-based decontaminants; and the like.

The liquid compositions of the present disclosure can also include other ingredients, such as organic co-solvents, surfactants, excipients, fillers, colorant, other active ingredients, ingredients that may be present in the other part, etc.

For example, the first liquid composition can include water in addition to the alcohol, even though it is listed as being present in the second liquid composition herein.

Examples of alcohols which can be used in the first liquid composition include but are limited to aliphatic alcohols and other carbon-containing alcohols, having from 1 to 24 carbons (C₁-C₂₄ alcohol). It is to be noted that “C₁-C₂₄ alcohol” does not necessarily imply only straight chain saturated aliphatic alcohols, as other carbon-containing alcohols can also be used within this definition, including branched aliphatic alcohols, alicyclic alcohols, aromatic alcohols, unsaturated alcohols, as well as substituted aliphatic, alicyclic, aromatic, and unsaturated alcohols, etc. In one embodiment, the aliphatic alcohols can be C₁ to C₅ alcohols including methanol, ethanol, propanol and isopropanol, butanols, and pentanols, due to their availability and lower boiling points. This being stated, polyhydric alcohols can also be used effectively in enhancing the decontaminant potency of the decontaminant solution of the present disclosure, as well as provide some degree of added stabilization. Examples of polyhydric alcohols which can be used in the present disclosure include but are not limited to ethylene glycol (ethane-1,2-diol), glycerin (or glycerol, propane-1,2,3-triol), sorbitol, and propane-1,2-diol. Other non-aliphatic alcohols may also be used including but not limited to phenols and substituted phenols, erucyl alcohol, ricinolyl alcohol, arachidyl alcohol, capryl alcohol, capric alcohol, behenyl alcohol, lauryl alcohol (1-dodecanol), myristyl alcohol (1-tetradecanol), cetyl (or palmityl) alcohol (1-hexadecanol), stearyl alcohol (1-octadecanol), isostearyl alcohol, oleyl alcohol (cis-9-octadecen-1-ol), palmitoleyl alcohol, linoleyl alcohol (9Z,12Z-octadecadien-1-ol), elaidyl alcohol (9E-octadecen-1-ol), elaidolinoleyl alcohol (9E,12E-octadecadien-1-ol), linolenyl alcohol (9Z,12Z,15Z-octadecatrien-1-ol), elaidolinolenyl alcohol (9E,12E,15-E-octadecatrien-1-ol), combinations thereof, and the like.

In some embodiments, for practical considerations, methanol, ethanol, and denatured alcohols (mixtures of ethanol and smaller amounts of methanol, and optionally, minute amounts of benzene, ketones, acetates, etc.) can often be used because of their availability and cost. Glycerol or sorbitol can also be used in some embodiments. If the desire is to provide a food grade composition, then alcohols can be selected that satisfy this requirement. When considering the amount of alcohol to use, one skilled in the art can stay within the above-described ranges, or modify these ranges for a particular application, considering such things as whether alcohol selected for use is polyhydric, whether the alcohol is food grade, mixtures of alcohols, etc.

Regarding the transition metal present in the first liquid composition, and ultimately in the decontaminant solution, the metal can be in ionic form (e.g. disassociate metal salt, metal ions from elemental metal, etc.) and/or in colloidal form. In one specific embodiment, the transition metal can be in a sub-micron form (i.e. dispersion of less than 1 μm metal colloidal particles). However, larger colloidal transition metal particles can also be used in certain applications. Typical transition metals that are desirable for use include Group VI to Group XI transition metals, and more preferably, can include Group X to Group XI transition metals. Alloys including at least one metal from the Group VI to Group XI metals can also be used. As shown below in the examples, some alloys can enhance or increase the decontaminant potency of the present disclosure. It is recognized that any of these metals will typically be oxidized to the corresponding cation in the presence of a peroxygen. However, with colloidal metals, typically, the surface is usually more susceptible to such oxidation. Further, when colloidal metals are dispersed in a colloidal solution, there is often an amount of the metal in ionic or salt form that is also present in the suspension solution. For example, colloidal silver may include a certain percentage of silver salt or ionic silver in solution, e.g., 10% to 90% by weight of metal content can be ionic based on the total metal content. This being stated, certain metals for use in accordance with embodiments of the present disclosure are ruthenium, rhodium, osmium, iridium, palladium, platinum, copper, gold, silver, manganese, zinc, alloys thereof, and mixtures thereof. Silver works well, but metal choice can be dependent to some degree on the application, the levels of destruction desired or required, the substrate that is being decontaminated, etc. For example, a piece of equipment of a certain material may be more appropriate for a different metal choice than silver.

It is also noted that any of these embodiments can often also benefit from the use of alloys. For example, certain combinations of metals in an alloy may provide benefits that are related more to other consideration, such as solution stability, substrate to be cleaned, etc. Examples of transition metal alloys for use in the present disclosure include but are not limited to copper-silver alloys, silver-manganese alloys, iron-copper alloys, chromium-silver alloys, gold-silver alloys, magnesium-silver alloys, germanium-silver alloys, and the like.

Exemplary colloidal silvers that can be used in the first liquid composition include those sold by Solutions IE, Inc. under the trade names CS Plus and CS Ultra. Other colloidal silver products that can be used as the silver source include ASAP, Sovereign Silver, Silver Max, and the like. In one embodiment, the colloidal particles used in the present disclosure can have a particle size range of from 0.001 μm to 1.0 μm. In another embodiment, the colloidal transition metal particles can have a size range of from 0.030 μm to 0.5 μm. In still another embodiment the average particle size is 0.35 μm to 0.45 μm. If used in ionic form, silver salts can include but are not limited to silver nitrate, silver acetate, silver citrate, silver oxide, and/or silver carbonate. Though many colloidal silver solutions or ionic silver solutions that are functional for use in the formulations of the present disclosure can be used, in one embodiment, it can be desirable to use RO water as the suspension medium for the colloidal and/or ionic silver that is mixed with the other ingredients. In a more detailed aspect, the RO water can also be distilled, resulting in 18-20 MΩ water, though this is not required.

The peroxygen component of the second liquid composition, and ultimately the decontaminant solution, can be a single compound or a combination of multiple peroxygen compounds or peroxygen forming compounds. In one embodiment, the peroxygen can be any aliphatic or aromatic peracid (or peroxyacid) that is functional for decontaminant purposes in accordance with embodiments of the present disclosure. While any functional peroxyacid can be used, peroxyacids containing from 1 to 7 carbons are the most practical for use. These peroxyacids can include, but not be limited to, peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, and/or peroxybenzoic acid. The peroxyacid used in the present disclosure can be prepared using any method known in the art. When the peroxyacid is prepared from an acid and hydrogen peroxide, the resultant mixture contains both the peroxyacid and the corresponding acid that it is prepared from. For example, in embodiments that utilize peroxyacetic acid, the presence of the related acid (acetic acid) provides stability to the mixture, as the reaction is an equilibrium between the acid, hydrogen peroxide, and the peroxyacid and water, as follows:

H₂O₂+CH₃COOH [symbol] CH₃COO—OH+H₂O

Peracid salts, such as salts of the above listed peracids, can also be included as the peroxygen component of the decontaminant solutions. Non-limiting examples of such salts include permanganates, perborates, perchlorates, peracetates, percarbonates, persulphates, and the like. The salts can be used alone or in combination with each other or other peroxygen compounds to form the peroxygen component of the disclosure.

In another embodiment, the peroxygen component of the disclosure can include a peroxide compound. While hydrogen peroxide is considered to be a desirable peroxide for use in accordance with embodiments of the present disclosure, other peroxides can also be used, such as metal peroxides and peroxyhydrates. The metal peroxides that can be used include, but are not limited to, sodium peroxide, magnesium peroxide, calcium peroxide, barium peroxide, and/or strontium peroxide. Other salts (for example sodium percarbonate) have hydrogen peroxide associated therewith much like waters of hydration, and these could also be considered to be a source of hydrogen peroxide, thereby producing hydrogen peroxide in situ. As mentioned above, the peroxides can be used alone or in combination with other peroxygen compounds to form the peroxygen component of the present disclosure. In one embodiment the peroxygen is a peracid and a peroxide.

Once the decontaminant solution of the present disclosure is formed using the system, it can be used to decontaminate any number of surfaces or instruments/equipment using any number of contacting methods. For example, the decontaminant solution can be used as a liquid dispersion bath for objects such as instruments or as a spray for applying to less mobile objects. In other words, any application method known by those skilled in the art can be utilized in accordance with embodiments of the present disclosure. Another possible application or method of use for the decontaminant solution includes, without limitation, use with a wipe where the liquid dispersion is applied to a fabric or fabric-like material for easy application without the need for spray or other application methods. In other words, any application method known by those skilled in the art can be utilized in accordance with embodiments of the present disclosure.

Additionally, though the decontaminant solution of the present disclosure is described generally as a decontaminant for destroying genetic material, it is recognized that there are many possible applications including its use as a disinfectant, sterilant, or sanitizer. For example, without limitation, the decontaminant solution of the present disclosure can be used to kill bacteria, spores, viruses, parasites, funguses, and molds that may also be present in the laboratory environment. As described, this composition can be used against all of these types of organisms with relative to complete safety to humans and other mammals.

### EXAMPLES

The following examples illustrate the embodiments of the disclosure that are presently best known. However, it is to be understood that the following are only exemplary or illustrative of the application of the principles of the present disclosure. Numerous modifications and alternative compositions, methods, and systems may be devised by those skilled in the art without departing from the spirit and scope of the present disclosure. The appended claims are intended to cover such modifications and arrangements. Thus, while the present disclosure has been described above with particularity, the following examples provide further detail in connection with what are presently deemed to be the most practical and preferred embodiments of the disclosure.

### Example 1

### Preparation of Decontaminant Solution

An aqueous decontaminant composition is prepared in accordance with embodiments of the present disclosure, which includes the following ingredients in approximate amounts: 85 wt % distilled water containing 600 ppm by weight colloidal silver; 9 wt % ethanol; and 6 wt % peroxyacetic acid. To the composition is added a small amount, i.e. <3 wt % based on the aqueous composition as a whole, of hydrogen peroxide to stabilize the peroxyacetic acid. It is noted that there will be less than 600 ppm by weight of the colloidal silver when based on the aqueous vehicle content as a whole.

### Example 2

### Preparation of Decontaminant Solution

An aqueous decontaminant composition is prepared in accordance with embodiments of the present disclosure, which includes the following ingredients in approximate amounts: 85 wt % distilled water containing 600 ppm by weight colloidal silver; 9 wt % isopropanol; and 6 wt % peroxypropanoic acid. To the composition is added a small amount of sodium peroxide to stabilize the peroxypropanoic acid. It is noted that there will be less than 600 ppm by weight of the colloidal silver when based on the aqueous vehicle content as a whole.

### Example 3

### Preparation of Decontaminant Solution

An aqueous decontaminant composition is prepared in accordance with embodiments of the present disclosure, which includes the following ingredients in approximate amounts: 75 wt % RO water (reverse osmosis water) containing 1500 ppm by weight colloidal silver; 15 wt % ethanol; and 10 wt % peroxyacetic acid. To the composition is added a small amount of hydrogen peroxide and acetic acid to the solution to stabilize the peracetic acid. It is noted that there will be less than 1500 ppm by weight of the colloidal silver when based on the aqueous vehicle content as a whole.

### Example 4

### Preparation of Decontaminant Solution

An aqueous decontaminant composition is prepared in accordance with embodiments of the present disclosure, which includes the following ingredients in approximate amounts: 75 wt % distilled water containing 10000 ppm by weight colloidal silver; 20 wt % denatured alcohol; and 5 wt % peroxyformic acid. Small amounts of hydrogen peroxide and formic acid are also added to the composition as a whole to stabilize the peroxyformic acid. It is noted that there will be less than 10000 ppm by weight of the colloidal silver when based on the aqueous vehicle content as a whole.

### Example 5

### Preparation of Decontaminant Solution

An aqueous decontaminant composition is prepared in accordance with embodiments of the present disclosure, which includes the following ingredients in approximate amounts: 85 wt % distilled water containing 80 ppm by weight colloidal silver; 9 wt % ethanol; and 6 wt % peroxyacetic acid. To the composition is added a small amount, i.e. <3 wt % based on the aqueous composition as a whole, of hydrogen peroxide to stabilize the peroxyacetic acid. It is noted that there will be less than 80 ppm by weight of the colloidal silver when based on the aqueous vehicle content as a whole.

### Example 6

### Preparation of Decontaminant Solution

An aqueous disinfectant composition is prepared in accordance with embodiments of the present invention, which includes the following ingredients in approximate amounts: 9 wt % isopropanol; 1.3 wt % peroxypropanoic acid (from a 6 wt % solution); less than 3 wt % of a peroxide, e.g., sodium peroxide, to stabilize the peroxypropanoic acid; and the balance being water containing 600 ppm ionic silver. It is noted that there will be less than 600 ppm by weight of the ionic silver when based on the aqueous vehicle content as a whole.

### Example 7

### Preparation of Decontaminant Solution Using Two-Part System

A two-part decontaminant system is provided. The first liquid composition of the system includes a solution of 20 parts by weight glycerol, 29.97 parts water and 0.03 parts colloidal silver (600 ppm). The second liquid composition includes, by weight, 1.3 parts peracetic acid and 48.7 parts water. The two components are kept separate until immediately before the decontaminant is desired for use. The decontaminant solution is made by mixing the two components at about a 1:1 (first:second) weight ratio to yield a composition having about 1.3 wt % peracetic acid and about 300 ppm silver. In this embodiment, less than 3 wt % of hydrogen peroxide can be added to further stabilize the system. This decontaminant solution can be used effectively to disinfect and sterilize a variety of surfaces.

### Example 8

### Preparation of Decontaminant Solution Using Two-Part System

A two-part decontaminant system is provided. The first liquid composition of the system includes a solution of about 10 parts by weight glycerol and about 81 parts by weight of a silver hydrosol (300 ppm colloidal silver). The second liquid composition of the system is includes an aqueous solution of 15 wt % peracetic acid in water. The two components are kept separate until immediately before the decontaminant is desired for use. The two components are combined at a weight ratio of 91:9 (first:second), yielding a solution having about 1.3 wt % peracetic acid. This decontaminant solution can be used effectively to disinfect and sterilize a variety of surfaces. It is noted that there will be less than 300 ppm by weight of the colloidal silver when based on the resultant decontaminant composition as a whole. This decontaminant solution can be used effectively to disinfect and sterilize a variety of surfaces.

### Example 9

### Preparation of Decontaminant Solution Using Two-Part System

A two-part decontaminant system is provided. The liquid composition of the system includes a solution of about 10 parts by weight glycerol and about 87 parts by weight of a silver hydrosol (800 ppm colloidal silver). The second liquid composition of the system is an aqueous solution of 15 wt % peracetic acid. The two components are kept separate until immediately before the decontaminant is desired for use. The decontaminant solution is made by mixing the two components at about a 97:3 (first:second) weight ratio to yield a composition having about 0.4 wt % peracetic acid. It is noted that there will be less than 300 ppm by weight of the colloidal silver when based on the resultant decontaminant composition as a whole. This decontaminant solution can be used effectively to disinfect and sterilize a variety of surfaces.

### Example 10

### Preparation of Concentrated Decontaminant Solution Using Two-Part System

A two-part decontaminant system is provided. The first liquid composition of the system is a solution of a silver alcosol (alcohol/3800 ppm silver). The second liquid composition of the system is an aqueous solution of 15 wt % peracetic acid. The two components are kept separate until immediately before the decontaminant is desired for use, and are admixed at a 17:13 (first:second) weight ratio. This resultant decontaminant solution can be further diluted using water. For example, 0.6 liters of the resultant decontaminant solution can be mixed with 2.4 liters of water to yield 3 liters of the decontaminant solution having 1.3 wt % PAA. This decontaminant solution can be used effectively to disinfect and sterilize a variety of surfaces.

### Example 11

### Preparation of Decontaminant Solution Using Two-Part System

A two-part decontaminant system is provided. The first liquid composition includes, by weight, 9 parts ethanol, 40.9 parts water, and 0.1 part silver (2,000 ppm). The second liquid composition includes, by weight, 1.3 parts peroxypropanoic acid and 48.7 parts water. The two components are kept separate until immediately before the decontaminant is desired for use. The decontaminant solution is made by mixing the two components at about a 1:1 (first:second) weight ratio to yield a composition having about 1.3 wt % peroxypropanoic acid and about 1,000 ppm silver. In this embodiment, less than 3 wt % of hydrogen peroxide can be added to further stabilize the system. This decontaminant solution can be used effectively to disinfect and sterilize a variety of surfaces.

### Example 12

### Preparation of Decontaminant Solution Using Two-Part System

A two-part decontaminant system is provided. The first liquid composition includes, by weight, 20 parts denatured alcohol, 29.45 parts water, and 0.05 parts silver and copper alloy (1,000 ppm). The second liquid composition is includes, by weight, 3 parts percitric acid and 47 parts water. The two components are kept separate until immediately before the decontaminant is desired for use. The decontaminant solution is made by mixing the two components at about a 1:1 (first:second) weight ratio to yield a composition having about 3 wt % percitric acid and about 500 ppm silver. In this embodiment, less than 3 wt % of hydrogen peroxide can be added to further stabilize the system. This decontaminant solution can be used effectively to disinfect and sterilize a variety of surfaces.

### Example 13

### Preparation of Decontaminant Solution Using Two-Part System

A two-part decontaminant system is provided. The first liquid composition included 0.015 wt % silver, 0.0004 wt % sorbitol, 10 wt % ethanol, and the balance water. The second liquid composition is included 22 wt % hydrogen peroxide, 15 wt % peroxyacetic acid, 15 wt % acetic acid, and the balance water. The two components are kept separate until immediately before the decontaminant is desired for use, though after activation, the composition can continue to be effective for several weeks. In this example, it is noted that activation of the resultant composition occurs by pouring the entire contents of the second liquid composition containing a premeasured 37.8 mL for gallon size (10.0 mL for liter) into the first liquid composition containing a premeasured 3,747.6 mL for gallon size (990.0 mL for liter) to achieve a 99:1 mixed volume ratio, which can be followed by agitating the combined solution for 15 seconds.

### Example 14

### Reduction of Genetic Material from Biological Samples

A study was conducted to evaluate the effectiveness of the admixed two part composition of Example 13 (hereinafter “Example 13 composition”) as an alternative to bleach for sterilizing laboratory work surfaces and instrumentation. Specifically, genetic material from body fluids, as well as extracted DNA and amplified DNA were evaluated for overall reduction. Clorox® Regular Bleach was used as a comparison decontaminant to the Example 13 composition. Unlike bleach, the Example 13 composition has no toxic fumes, no emissions into the environment, and no corrosive chemicals that damage equipment or tools. Once activated, the Example 13 composition (two parts mixed together) is expected to last for a period of weeks or even months. Conversely, diluting pure bleach requires daily labor to make new batches, since bleach breaks down and loses its effectiveness within 24 hours of being diluted.

The following method was used to evaluate the effectiveness of the decontaminants without the mechanical action of wiping, e.g., destruction of DNA without wiping. Samples of blood, extracted DNA, and amplified DNA were added to the decontaminants and allowed to remain exposed to them for controlled periods of times. At the end of this exposure, the samples were assessed to determine how much DNA remained.

To determine the ability of the decontaminants to destroy DNA in a blood sample, an extracted DNA sample, or an amplified DNA sample, ratios of one part sample volume to five parts decontaminant composition volume, respectively, was used for both bleach and the Example 13 composition.

a) Blood

A 10% bleach solution and the Example 13 composition were both allowed to remain in contact with various blood samples for 5 minutes. After these times had been reached, the samples were put through the quantitation process to determine if there was any reduction in the DNA content of any of the samples. In some cases, since there was a possibility that the concentration of DNA would be less simply by the addition of liquid, which would dilute the sample, a TE buffer was added to a separate set of “control” extracts in the same concentrations: 1 part to 5 parts (v/v) and 1 part to 10 parts (v/v). In each case, the samples that were decontaminated with the Example 12 composition provided a greater reduction in DNA compared to 10% bleach solutions. To illustrate, Table 1 below sets forth data for comparative reduction in DNA with initial blood samples of 20 μl of blood to 100 μl of decontaminant (1:5 v/v), or with initial blood samples of 20 μl of blood to 200 μl of decontaminant (1:10 v/v).

TABLE 1
Results of the Blood with No Wiping (1:5 v/v; 1:10 v/v) at 20 μl
of blood

| | | Bleach | Example 12 |
|---|---|---|---|
| | TE Buffer | solution | composition |
| Decontaminant | (ng/μl DNA) | (ng/μl DNA) | (ng/μl DNA) |
| Sample 1a - 20 μl of blood to | 12.60 | 5.45 | 0.46 |
| 100 μl Decontaminant or | | | |
| Buffer | | | |
| Sample 1b - 20 μl of blood to | 13.50 | 7.42 | 0.81 |
| 100 μl Decontaminant or | | | |
| Buffer | | | |
| Sample 1c - 20 μl of blood to | 15.70 | 7.30 | 0.35 |
| 100 μl Decontaminant or | | | |
| Buffer | | | |
| Sample 2a - 20 μl of blood to | 12.60 | 1.78 | 0.26 |
| 200 μl Decontaminant or | | | |
| Buffer | | | |
| Sample 2b - 20 μl of blood to | 13.50 | 6.63 | 0.30 |
| 200 μl Decontaminant or | | | |
| Buffer | | | |
| Sample 2c - 20 μl of blood to | 15.70 | 1.78 | 0.30 |
| 200 μl Decontaminant or | | | |
| Buffer | | | |

As can be seen in Table 1, on average, about a 2-fold reduction in DNA was obtained using bleach over the control (TE Buffer), and average of about a 30-fold reduction in DNA was obtained using the Example 12 composition at a 1:5 v/v ratio of blood to decontaminant composition. Furthermore, on average, about a 7-fold reduction in DNA was obtained using bleach over the control (TE Buffer), and an average of about a 47-fold reduction in DNA was obtained using the Example 12 composition at a 1:10 v/v ratio of blood to decontaminant composition. Thus, as an improvement over bleach, the composition of Example 12 specifically was effective in destroying the DNA, even when encased within cells like blood.

b) Amplified DNA and Extracted DNA

With specific reference to non-encased DNA, i.e. amplified DNA or extracted DNA, both bleach and the Example 12 composition at similar volume ratios described in Table 1 provided a negative quantitation result, i.e. successful destruction of the extracted DNA.

c) Benefits of Mechanical Wiping After Decontamination

In order to avoid the possibility of a decontaminant composition generating possible false readings during subsequent genetic testing after decontamination, it is sometimes prudent to conduct a mechanical wiping step after or coincident with decontamination. Though not required, this can provide desired removal of decontaminant composition prior to additional genetic testing in a given lab environment. Proper cleanup can include allowing the decontaminant composition it to remain in contact with the surface for an appropriate amount time to destroy genetic material. Since any decontaminant does not guarantee the removal of all DNA, the action of wiping the surface can be performed, perhaps followed by a secondary wiping with water. Removal of decontaminant reduces the chance of generating negative effects on the PCR, electrophoresis, or other similar processes.

While the disclosure has been described with reference to certain preferred embodiments, those skilled in the art will appreciate that various modifications, changes, omissions, and substitutions can be made without departing from the spirit of the disclosure. It is therefore intended that the disclosure be limited only by the scope of the appended claims.

What is claimed is:

1. A method of destroying genetic material in laboratory environment, comprising:
contacting a surface in a laboratory that is contaminated with genetic material with a decontaminant composition comprising a transition metal or alloy thereof, an alcohol, a peroxygen, and water; and
maintaining contact between the decontaminant composition and the surface for a sufficient period of time to provide at least a reduction in genetic material on the surface.

2. The method of claim 1, wherein the surface is hard surface.

3. The method of claim 1, wherein the surface includes laboratory equipment.

4. The method of claim 1, wherein the surface includes collection, storage, or transportation equipment.

5. The method of claim 1, wherein the genetic material is present in a bodily fluid.

6. The method of claim 6, wherein the bodily fluid is blood.

7. The method of claim 6, wherein the bodily fluid is semen.

8. The method of claim 6, wherein the bodily fluid comprises saliva or mucous.

9. The method of claim 1, wherein the genetic material is present in skin or hair.

10. The method of claim 1, wherein the genetic material comprises DNA.

11. The method of claim 10, wherein the DNA comprises extracted DNA.

12. The method of claim 10, wherein the DNA comprises amplified DNA.

13. The method of claim 1, wherein the genetic material comprises RNA.

14. The method of claim 1, further comprising the step of wiping the surface after the contacting step, but before the maintaining step.

15. The method of claim 1, further comprising the step of wiping the surface after the maintaining step.

16. The method of claim 1, further comprising the step of wiping after achieving at least a three-fold reduction in genetic material on the surface.

17. The method of claim 1, wherein the step of maintaining contact between the decontaminant composition and the surface provides at least a three-fold reduction in genetic material on the surface.

18. The method of claim 1, wherein the step of maintaining contact between the decontaminant composition and the surface provides at least a ten-fold reduction in genetic material on the surface.

19. The method of claim 1, wherein the step of maintaining contact between the decontaminant composition and the surface provides at least a forty-fold reduction in genetic material on the surface.

20. The method of claim 1, wherein the decontaminant composition is prepared by admixing at least two parts together in accordance with the preliminary step of admixing a first liquid composition and a second liquid composition to form the decontaminant composition, the first liquid composition comprising the transition metal or alloy thereof and the alcohol, the second liquid composition comprising the peroxygen and the water.

21. The method of claim 20, wherein the admixing step further includes adding additional water to at least one of the first liquid composition, the second liquid composition, and the decontaminant composition in preparation for use.

22. The method of claim 20, wherein the first liquid composition also includes water.

23. The method of claim 20, wherein the alcohol is present in the first liquid composition at from about 0.005 wt % to 99.99 wt %.

24. The method of claim 20, wherein the alcohol is present in the first liquid composition at from 0.1 wt % to 50 wt %.

25. The method of claim 20, wherein the transition metal or alloy thereof is present in the first liquid composition at from 0.0005 ppm to 100,000 ppm by weight.

26. The method of claim 20, wherein the transition metal or alloy thereof is present in the first liquid composition at from 0.01 ppm to 20,000 ppm by weight.

27. The method of claim 20, wherein the peroxygen is present in the second liquid composition at from 0.01 wt % to 30.0 wt %.

28. The method of claim 1, wherein the alcohol is a C₁-C₂₄ alcohol.

29. The method of claim 1, wherein the alcohol is a polyhydric alcohol.

30. The method of claim 29, wherein the polyhydric alcohol includes glycerol or sorbitol.

31. The method of claim 1, wherein the transition metal or alloy thereof is selected from the group consisting of ruthenium, rhodium, osmium, iridium, palladium, platinum, copper, gold, silver, manganese, zinc, alloys thereof, and mixtures thereof.

32. The method of claim 1, wherein the transition metal or alloy thereof is a colloidal transition metal or alloy thereof.

33. The method of claim 32, wherein the colloidal transition metal or alloy thereof is colloidal silver.

34. The method of claim 32, wherein the colloidal transition metal or alloy thereof has an average particle size of from 0.030 μm to 0.5 μm.

35. The method of claim 1, wherein the transition metal or alloy thereof is an ionic transition metal.

36. The method of claim 1, wherein the transition metal or alloy thereof is present in the decontaminant composition at from 15 ppm to 1,500 ppm by weight.

37. The method of claim 1, wherein the peroxygen includes a peracid.

38. The method of claim 37, wherein the peracid is selected from the group consisting of peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, peroxybenzoic acid, and mixtures thereof.

39. The method of claim 1, wherein the peroxygen is present in the decontaminant composition at from 0.1 wt % to 10 wt %.

40. The method of claim 1, wherein the peroxygen is present in the decontaminant composition at from 0.2 wt % to 5 wt %.

41. The method of claim 1, wherein the peroxygen is a peroxide.

42. The method of claim 1, wherein the peroxygen includes a peracid and a peroxide.

43. The method of claim 1, wherein the decontaminant solution is substantially free of aldehydes, chlorine and bromine-containing compositions, iodophore-containing compositions, phenolic-containing compositions, and quaternary ammonium-containing compositions.

44. The method of claim 1, wherein the decontaminant composition is applied to the surface at least at a 1:1 weight ratio of decontaminant composition to genetic material sample.

45. The method of claim 1, wherein the decontaminant composition is applied to the surface at least at a 5:1 weight ratio of decontaminant composition to genetic material sample.

46. The method of claim 1, wherein the decontaminant composition is applied to the surface at least at a 10:1 weight ratio of decontaminant composition to genetic material sample.

47. The method of claim 1, wherein the laboratory is a forensics laboratory.

48. The method of claim 1, wherein the laboratory is a medical laboratory.

49. The method of claim 1, wherein the laboratory is a mobile laboratory.

50. The method of claim 1, wherein the laboratory includes a lab on a chip.
